(19) United States
(12) Patent Application Publication
(10) Pub. No.: US 2025/0049636 A1
(43) Pub. Date: Feb. 13, 2025

(54) COLLECTION KIT AND COLLECTION METHOD

(71) Applicant: TERUMO KABUSHIKI KAISHA, Tokyo (JP)

(72) Inventors: Mayu KATAGIRI, Fujinomiya-shi Shizuoka (JP); Hiroshi OKABE, Fujinomiya-shi Shizuoka (JP)

(21) Appl. No.: 18/931,507

(22) Filed: Oct. 30, 2024

Related U.S. Application Data
(63) Continuation of application No. PCT/JP2023/020204, filed on May 31, 2023.

(30) Foreign Application Priority Data
May 31, 2022 (JP) 2022-088611

(51) Int. Cl. A61J 1/12; A61J 1/16; A61J 1/20

(52) U.S. Cl. CPC A61J 1/12; A61J 1/16; A61J 1/2006; A61J 1/2058; A61J 1/2075

(57) ABSTRACT

A collection kit includes an inlet tube to which a medical bag accommodating an object to be collected is connected; and a collection bag that is connected to a downstream side of the inlet tube, has a plurality of accommodation chambers formed by overlapping and welding two flexible sheets, and accommodates a predetermined amount of the object to be collected in each of the accommodation chambers. The collection bag integrally includes a tubular connection flow path connecting the adjacent accommodation chambers.

### CROSS-REFERENCE TO RELATED APPLICATIONS

The present disclosure is a continuation of and claims benefit to PCT/JP2023/020204 filed on May 31, 2023, entitled “COLLECTION KIT AND COLLECTION METHOD” which claims priority to Japanese Patent Application No. 2022-088611 filed on May 31, 2022. The entire disclosure of the applications listed above are hereby incorporated herein by reference, in their entireties, for all that they teach and for all purposes.

### BACKGROUND

The present disclosure relates to a collection kit and a collection method for collecting a blood product for inspection.

Blood products include a red blood cell product, a plasma product, a platelet product, and a whole blood product. Blood products containing components required by patients are used for blood transfusion. The blood products are stored and transported in the state of being accommodated in medical bags. In order to ensure safety of the blood products, a small amount of a sample may be collected to perform a culture test. In the culture test, the sample is collected in a culture bottle, and the culture bottle is placed in an environment suitable for bacterial growth to detect the presence or absence of a pathogen.

Among the blood products, a test for the platelet product is performed by the following procedure. A collection bag having a small volume is connected to a medical bag accommodating the platelet product (hereinafter, referred to as a platelet bag). Thereafter, a part of the platelet product in the platelet bag is transferred to the collection bag. The collection bag is then disconnected and separated from the platelet bag.

Next, the collection bag is conveyed to a clean bench. At the clean bench, the platelet product in the collection bag is infused (e.g., injected) into a blood culture bottle. Prior to the infusion, a tube of the collection bag is connected to a tube extending from a sample collection tube. A prescribed amount of the platelet product is transferred from the collection bag to the sample collection tube with reference to scales of the sample collection tube.

Thereafter, an operator operates a valve of the sample collection tube while visually observing the scales of the sample collection tube to transfer the prescribed amount of the platelet product from the sample collection tube to the blood culture bottle. In the culture test, anaerobic culture and aerobic culture are performed. Therefore, the platelet product is dispensed into a culture bottle used for the anaerobic culture and a culture bottle used for the aerobic culture. Thereafter, the culture bottles are set in a culturing apparatus and subjected to the culture test for a predetermined period.

One example of a collection device for collecting a sample from a medical bag is disclosed in U.S. Pat. No. 8,777,921, entitled “Sterile Sampling Methods and Apparatus.”

### BRIEF SUMMARY

From the perspective of further improving the safety of a platelet product, a culture test for a total number of the platelet product has been studied. Therefore, it is required to improve the work efficiency of the culture test of the platelet product. In addition, in the case of performing the culture test for the total number, it is desirable that an instrument for distributing the platelet product into culture bottles has a simple structure such that cost can be reduced.

It is an object of the present disclosure to solve the above issues and other problems.

According to at least one aspect of the present disclosure, a collection kit is provided including: an inlet tube to which a medical bag accommodating an object to be collected is connected; a collection bag which is connected to a downstream side of the inlet tube, includes a plurality of accommodation chambers formed by overlapping and welding two flexible sheets, and accommodates a predetermined amount of the object to be collected in each of the accommodation chambers; a plurality of outlet ports which are connected to the accommodation chambers, respectively, and allow the object to be collected to flow out from the accommodation chambers; and adapters which are connected to downstream sides of the outlet ports, respectively, and connect culture bottles and the outlet ports by the culture bottles being attached to the adapters, respectively, wherein the collection bag includes a connection flow path connecting the accommodation chambers adjacent to each other, and the connection flow path is integrally formed with the accommodation chambers by the two flexible sheets, and has a tubular portion formed in a tubular shape by cutting out the flexible sheets around the tubular portion between the accommodation chambers adjacent to each other.

In addition, according to another aspect of the present disclosure, a collection method is provided using the collection kit described above, the collection method including: a step of connecting the medical bag to the inlet tube; a step of introducing the object to be collected into the accommodation chambers of the collection bag through the inlet tube; a step of separating the medical bag from the inlet tube; a step of closing the tubular portion to prevent the object to be collected from moving between the plurality of accommodation chambers; and a step of connecting the culture bottles to the adapters and injecting the object to be collected into the culture bottles.

The collection kit and the collection method of the present disclosure, described above and herein, have a simple configuration, and thus can be manufactured at low cost and have excellent work efficiency.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a plan view of a collection kit according to a first embodiment of the present disclosure.

FIG. 2 is an explanatory view of a step of introducing a platelet product into the collection kit of FIG. 1.

FIG. 3 is an explanatory view of a step of introducing the platelet product introduced into first and second accommodation chambers of FIG. 1 into the first and second culture bottles.

FIG. 4 is a schematic explanatory view of a collection kit according to a second embodiment of the present disclosure.

FIG. 5A is a perspective view of the collection kit of FIG. 4.

FIG. 5B is a cross-sectional view taken along line VB-VB of FIG. 4.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Unless otherwise specified, all technical terms and scientific terms used herein have the same meaning that those skilled in the art commonly understand. All patents, applications and other publications and information cited herein are incorporated herein by reference in their entirety.

It is with respect to the above issues and other problems that the embodiments presented herein were contemplated.

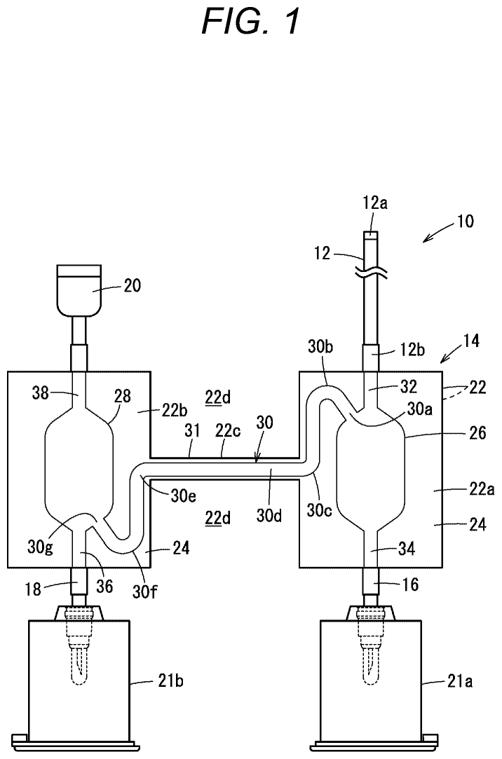

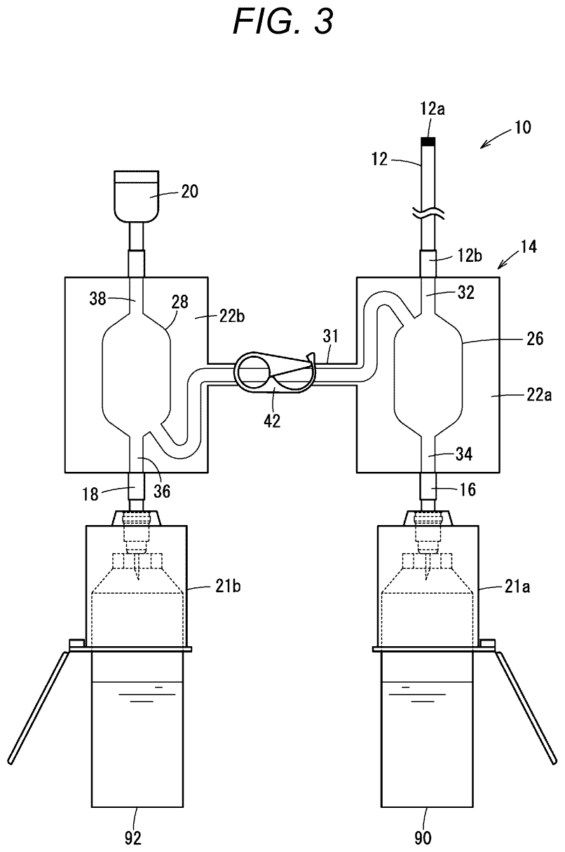

A collection kit 10 according to the present embodiment illustrated in FIG. 1 is used for, for example, a culture test for confirming the safety of a product at a business facility such as a blood center that manufactures (e.g., collects, separates, sorts, and/or otherwise processes) blood products. The culture test includes a culture of anaerobic bacteria and a culture of aerobic bacteria. Therefore, in the culture test, two bottles of a first culture bottle 90 (see, e.g., FIG. 3) for an anaerobic culture and a second culture bottle 92 (see, e.g., FIG. 3) for an aerobic culture are used. The collection kit 10 is used to collect a liquid sample (platelet product) and dispense the sample into the two culture bottles 90 and 92 each in a predetermined amount.

Although not particularly limited, an example in which a platelet product is used as a blood product will be described in accordance with embodiments of the present disclosure. As illustrated in FIG. 1, the collection kit 10 includes an inlet tube 12, a collection bag 14, a first sealing member 16, a second sealing member 18, an exhaust valve 20, a first adapter 21a, and a second adapter 21b. In addition, in the collection kit 10, a direction in which the inlet tube 12 and the exhaust valve 20 are located is referred to as an upper side or upward, and a direction in which the first adapter 21a and the second adapter 21b are located is referred to as a lower side or downward based on an arrangement state during use. In addition, a direction perpendicular to the vertical direction and along a plane of the collection bag 14 is referred to as a width direction, and a direction perpendicular to the plane of the collection bag 14 is referred to as a thickness direction.

The inlet tube 12 is, for example, a translucent medical tube made of a thermoplastic resin such as a vinyl chloride resin. The inlet tube 12 can be connected to or separated from another medical tube without exposing the inside thereof to outside air, for example, by an aseptic joining device, a tube sealer, or the like. The inlet tube 12 has an upstream first end 12a and a downstream second end 12b. The first end 12a is welded and sealed in the initial state. The second end 12b is connected to an upper end of the collection bag 14.

The collection bag 14 has two flexible sheets 22 overlapping each other in the thickness direction. The two flexible sheets 22 are welded and joined to one another at a plurality of welding portions 24 (e.g., weld regions). Each of the flexible sheets 22 includes a first region 22a for forming one accommodation chamber (a first accommodation chamber 26), a second region 22b for forming the other accommodation chamber (a second accommodation chamber 28), and a connection region 22c connecting the first region 22a and the second region 22b. The first region 22a has a rectangular shape having the same dimensions as the second region 22b in a plan view. The connection region 22c extends in the width direction and is integrally connected to the first region 22a and the second region 22b. Each of the flexible sheets 22 has a pair of cutout portions 22d obtained by cutting out the flexible sheet 22 in a rectangular shape on the upper side and the lower side of the connection region 22c. The connection region 22c is formed between the upper and lower cutout portions 22d, and is formed such that a dimension in the vertical direction is narrower than a dimension in the width direction. Although not particularly limited, the connection region 22c is located at the center in the vertical direction of the first region 22a and the second region 22b. In some embodiments, the cutout portions 22d may correspond to recesses that are formed in the material of the two flexible sheets 22 (e.g., prior to welding the two flexible sheets 22 together).

The collection bag 14 includes the first accommodation chamber 26, the second accommodation chamber 28, and a connection flow path 30 defined by the welding portions 24. The first accommodation chamber 26 is formed in the first regions 22a of the flexible sheets 22. The first accommodation chamber 26 accommodates the platelet product to be introduced into the first culture bottle 90. The first accommodation chamber 26 bulges in the thickness direction of the flexible sheets 22 in the initial state (state at the time of being provided as a product). In the first accommodation chamber 26, one of the flexible sheets 22 and the other flexible sheet 22 are separated from each other in the thickness direction. The first accommodation chamber 26 has a volume for accommodating a predetermined amount (for example, about 8 ml or about 10 ml) of the platelet product.

The collection bag 14 has an inlet port 32 connected to the first accommodation chamber 26 and a first outlet port 34. The inlet port 32 is connected to an upper end of the first accommodation chamber 26 of the collection bag 14. The inlet port 32 is connected to the second end 12b of the inlet tube 12, and allows the inlet tube 12 and the first accommodation chamber 26 to communicate with each other. The first outlet port 34 (e.g., outflow port) is connected to a lower end of the first accommodation chamber 26. The first outlet port 34 is connected to the first sealing member 16. The first adapter 21a is connected to the first outlet port 34 via the first sealing member 16.

The second accommodation chamber 28 is formed in the second regions 22b of the flexible sheets 22. The second accommodation chamber 28 accommodates the platelet product to be introduced into the second culture bottle 92. The second accommodation chamber 28 has the same shape as the first accommodation chamber 26 and accommodates the same volume of the platelet product.

The collection bag 14 has a second outlet port 36 and an exhaust port 38 which are connected to the second accommodation chamber 28. The second outlet port 36 (e.g., outflow port) is connected to a lower end of the second accommodation chamber 28 of the collection bag 14. The second outlet port 36 is connected to the second sealing member 18. The second adapter 21b is connected to the second outlet port 36 via the second sealing member 18. The exhaust port 38 is connected to an upper end of the second accommodation chamber 28. The exhaust valve 20 is connected to the exhaust port 38.

The connection flow path 30 is a flow path that connects the first accommodation chamber 26 and the second accommodation chamber 28. An upstream end 30a of the connection flow path 30 is connected to an upper portion of the first accommodation chamber 26. The connection flow path 30 extends obliquely upward from the end 30a and is curved at a top portion 30b to extend downward. The connection flow path 30 extends downward to the center in the vertical direction of the collection bag 14 on the downstream side of the top portion 30b, and is bent in the width direction at a bent portion 30c. The connection flow path 30 forms an intermediate portion 30d extending in the width direction in a direction away from the first accommodation chamber 26 on the downstream side of the bent portion 30c. A middle portion of the connection flow path 30 in a flow path direction is located at the center of the intermediate portion 30d in the width direction. The intermediate portion 30d extends along the connection regions 22c of the flexible sheets 22 and extends to the second regions 22b. The connection flow path 30 has a bent portion 30e on the downstream side of the intermediate portion 30d. The connection flow path 30 is bent downward at the bent portion 30e. The connection flow path 30 on the downstream side of the bent portion 30e is turned obliquely upward in a V shape at a folded portion 30f, so that a downstream end 30g located on the downstream side of the folded portion 30f is connected to a lower portion of the second accommodation chamber 28.

The connection flow path 30 bulges in the thickness direction in the initial state. In addition, the intermediate portion 30d of the connection flow path 30 forms a tubular portion 31 having a tubular shape in which a portion passing through the connection region 22c is elongated in the width direction. The tubular portion 31 is sealed by the welding portion 24 whose upper end and lower end are narrow, and the intermediate portion 30d of the connection flow path 30 can be sealed by attaching a clamp 42 through the cutout portions 22d. In addition, the tubular portion 31 can be cut in the sealed state using a tube sealer that cuts a tube while sealing a flow path by heating with high frequency waves or ultrasonic waves.

The first sealing member 16 seals the first outlet port 34 in the initial state to prevent passage of fluid between the first accommodation chamber 26 and the first adapter 21a. The first sealing member 16 is a cylindrical member having a breakable plug therein, and when an internal flow path performs an operation of bending the plug in the initial state, the plug is broken so that the internal flow path can be opened. The second sealing member 18 is a member similar to the first sealing member 16, and seals the second outlet port 36 in the initial state to prevent passage of fluid between the second accommodation chamber 28 and the second adapter 21b.

The exhaust valve 20 communicates with the second accommodation chamber 28 and the first accommodation chamber 26 through the exhaust port 38. The exhaust valve 20 internally has a filter that allows passage of gas and prevents passage of liquid. Although not particularly limited, the exhaust valve 20 may have a hydrophilic filter. When the exhaust valve 20 having the hydrophilic filter comes into contact with the platelet product after the first accommodation chamber 26 and the second accommodation chamber 28 are filled with the platelet product, pores of the filter are clogged by liquid, and thereafter, the exhaust valve 20 prevents the passage of gas and liquid. Such an exhaust valve 20 can prevent backflow of air into the second accommodation chamber 28.

As illustrated in FIG. 3, the first adapter 21a accommodates a neck portion of the first culture bottle 90. The first adapter 21a opens the first sealing member 16 by an internal needle tube penetrating a stopper of the first culture bottle 90, and then causes the first culture bottle 90 and the first accommodation chamber 26 to communicate with each other (e.g., fluidly communicate, etc.). The second adapter 21b is a member similar to the first adapter 21a, and opens the second sealing member 18 and then causes the second culture bottle 92 and the second accommodation chamber 28 to communicate with each other (e.g., fluidly communicate, etc.).

The collection kit 10 of the present embodiment is configured as described above. The collection kit 10 is used as follows.

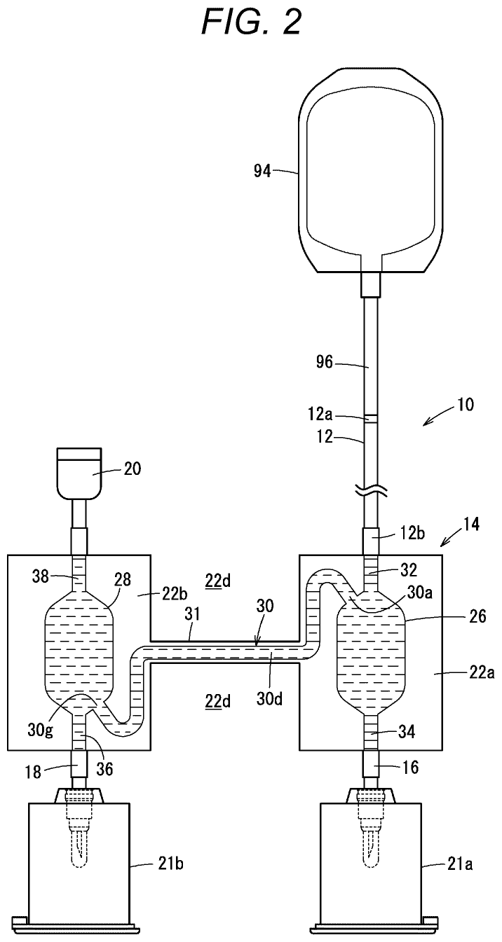

In the present embodiment, a collection method for dispensing the platelet product into the two culture bottles 90 and 92 using the collection kit 10 will be described. First, a platelet bag 94 is joined to the collection kit 10. As illustrated in FIG. 2, the platelet bag 94 is a medical bag accommodating the platelet product. The platelet bag 94 has a tube 96 for connection. The tube 96 is joined to the inlet tube 12. The tube 96 is joined to the inlet tube 12 without coming into contact with outside air using an aseptic joining device.

Next, the platelet product is introduced into the collection bag 14. In this step, in the collection kit 10 and the platelet bag 94, the platelet bag 94 is arranged above the collection bag 14. The first sealing member 16 and the second sealing member 18 of the collection kit 10 are maintained in a closed state.

The platelet product flows out of the platelet bag 94 by gravity and flows into the collection bag 14 through the inlet tube 12. The platelet product flowing into the collection bag 14 flows into the first accommodation chamber 26 through the inlet port 32. The platelet product is stored more and more in the first accommodation chamber 26, and a liquid level of the platelet product in the first accommodation chamber 26 rises. Air inside the first accommodation chamber 26 is pushed out by the platelet product and is discharged from the exhaust valve 20 through the connection flow path 30 and the second accommodation chamber 28.

When the first accommodation chamber 26 is filled with the platelet product, the platelet product flows into the upstream end 30a of the connection flow path 30. Thereafter, the platelet product flows through the connection flow path 30 from upstream to downstream, and flows into the second accommodation chamber 28 from the lower portion of the second accommodation chamber 28. As the inflow of the platelet product proceeds, a liquid level of the platelet product inside the second accommodation chamber 28 rises. Air in the second accommodation chamber 28 is discharged from the exhaust valve 20. The second accommodation chamber 28 is then filled with the platelet product. Furthermore, when the exhaust valve 20 comes into contact with the platelet product, the exhaust valve 20 is closed to prevent the inflow of the platelet product. As a result, the introduction of the platelet product into the collection bag 14 is completed. A predetermined amount (for example, about 8 ml) of the platelet product is accommodated in each of the first accommodation chamber 26 and the second accommodation chamber 28.

Thereafter, the platelet bag 94 is separated from the inlet tube 12 by a tube sealer (e.g., high frequency or ultrasonic sealer) or the like. The tube sealer separates the inlet tube 12 from the tube 96 of the platelet bag 94, and at the same time, seals the first end 12a of the inlet tube 12 by welding. The separation between the inlet tube 12 and the tube 96 is performed without exposing internal flow paths to the outside air.

The detached platelet bag 94 is stored until a culture test is completed, and then available for use. In addition, the collection kit 10 filled with the platelet product is carried into a clean bench.

Thereafter, as illustrated in FIG. 3, the first culture bottle 90 and the second culture bottle 92 are connected to the first adapter 21a and the second adapter 21b of the collection kit 10, respectively. Thereafter, the tubular portion 31 of the connection flow path 30 is closed by the clamp 42. As the tubular portion 31 is closed, fluid can be prevented from moving between the first accommodation chamber 26 and the second accommodation chamber 28. Note that the tubular portion 31 may be closed while being cut by a tube sealer. In addition, the tubular portion 31 may be closed by a method of welding the two flexible sheets 22 by sealing (also referred to as ultrasonic sealing or high frequency sealing) in which sealing is performed by melting a resin by heating with ultrasonic waves or high frequency waves (electromagnetic waves). In this case, it is not necessary to provide the clamp 42 in a circuit in advance.

Thereafter, an operation of opening the first sealing member 16 is performed, the platelet product in the first accommodation chamber 26 is sucked out due to the negative pressure of the first culture bottle 90, and a predetermined amount of the platelet product is introduced into the first culture bottle 90. In addition, an operation of opening the second sealing member 18 is performed, the platelet product in the second accommodation chamber 28 is sucked out due to the negative pressure of the second culture bottle 92, and a predetermined amount of the platelet product is introduced into the second culture bottle 92.

The first culture bottle 90 is detached from the first adapter 21a, and the second culture bottle 92 is detached from the second adapter 21b. Through the above steps, the collection of the platelet product using the collection kit 10 is completed.

As described above, the collection kit 10 of the present embodiment has a structure in which the collection bag 14 is integrally formed using the two flexible sheets 22, and thus can be manufactured at low cost. In addition, the number of times of joining and separation between the tubes can be minimized, the work efficiency is excellent. Furthermore, since it is not necessary to perform the work of measuring and taking the platelet product by eye according to the scale line, the work becomes easy and the measurement error can be reduced. In addition, the collection kit 10 can reliably separate the first accommodation chamber 26 and the second accommodation chamber 28 by closing the tubular portion 31 of the collection bag 14 with the clamp or the like, and can prevent the platelet product from moving between the first accommodation chamber 26 and the second accommodation chamber 28. Therefore, the collection kit 10 can suppress variations in the amount of the platelet product introduced into each of the first culture bottle 90 and the second culture bottle 92.

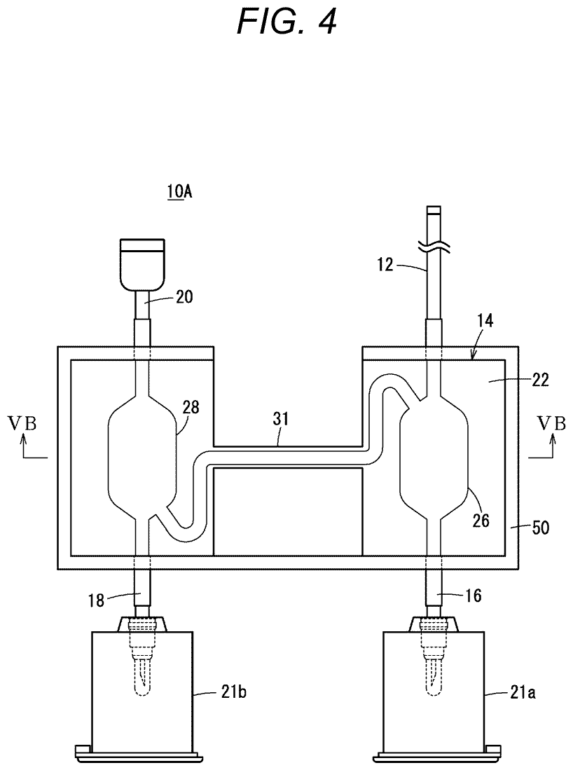

As illustrated in FIG. 4, a collection kit 10A in accordance with embodiments of the present disclosure may differ from the collection kit 10 illustrated in FIG. 1 in further including a frame member 50 that holds the collection bag 14. Note that the inlet tube 12, the collection bag 14, the exhaust valve 20, the first adapter 21a, and the second adapter 21b in the collection kit 10A of FIG. 4 are similar, if not identical, to those of the collection kit 10, and thus the detailed description thereof will be omitted.

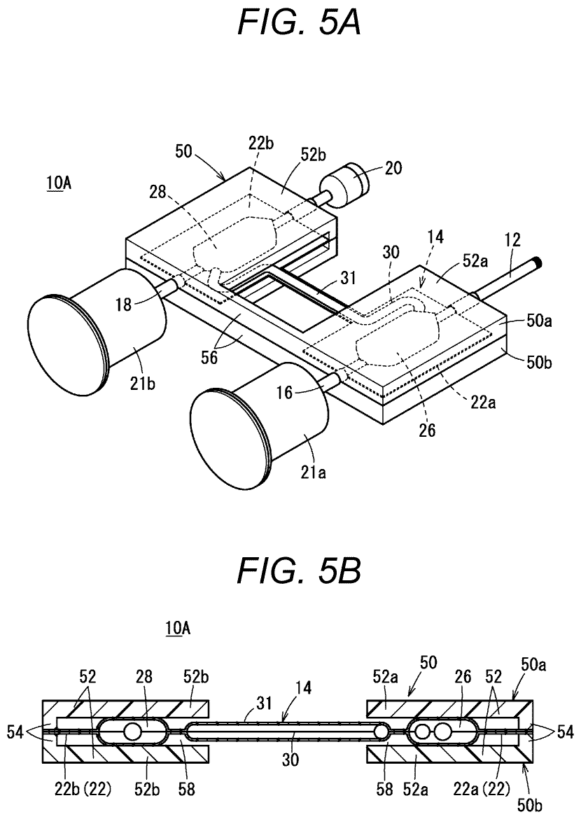

As illustrated in FIG. 5A, the collection kit 10A includes the frame member 50 that holds the collection bag 14. The frame member 50 includes a first frame member 50a and a second frame member 50b. The first frame member 50a is arranged adjacent to one side of the collection bag 14 in the thickness direction. The second frame member 50b is arranged adjacent to the other side of the collection bag 14 in the thickness direction. The frame member 50 has a structure in which the collection bag 14 is sandwiched between the first frame member r and the second frame member 50b in the thickness direction.

As illustrated in FIGS. 5A and 5B, the first frame member 50a includes an abutment plate 52, a frame body 54, and a connection portion 56. The abutment plate 52 includes a rectangular first flat plate portion 52a formed in a portion corresponding to the first region 22a and a second flat plate portion 52b formed in a portion corresponding to the second region 22b. In the first flat plate portion 52a, the frame body 54 is formed over three sides except for a side facing the center in the width direction. In addition, the frame body 54 is formed over three sides excluding a side facing the center in the width direction in the second flat plate portion 52b. The frame body 54 bulges in the thickness direction with respect to the first flat plate portion 52a and the second flat plate portion 52b. The frame body 54 has a groove to allow passage of a pipe connected to the inlet tube 12, the first sealing member 16, the second sealing member 18, and the exhaust valve 20 in a predetermined part. The first flat plate portion 52a and the second flat plate portion 52b are connected to each other via the connection portion 56. The connection portion 56 is located, for example, at a lower end of the first frame member 50a. The connection portion 56 is away from the tubular portion 31 of the collection bag 14 and is arranged at a position that does not hinder attachment of the clamp 42 or the like to the tubular portion 31.

The first frame member 50a forms recesses 58 in portions surrounded by the frame body 54. The recesses 58 accommodate portions in the thickness direction of the first accommodation chamber 26 and the second accommodation chamber 28, respectively.

The second frame member 50b is formed to be symmetrical with the first frame member 50a in the thickness direction, and includes the abutment plate 52, the frame body 54, and the connection portion 56 similar to those of the first frame member 50a. The second frame member 50b has the recesses 58 in areas surrounded by the frame body 54, and accommodates the other portions in the thickness direction of the first accommodation chamber 26 and the second accommodation chamber 28, respectively.

The abutment plate 52 of the first frame member 50a and the abutment plate 52 of the second frame member 50b abut on each other (e.g., contact one another) so as to sandwich the first accommodation chamber 26 and the second accommodation chamber 28 in the thickness direction, and prevent the first accommodation chamber 26 and the second accommodation chamber 28 from expanding (e.g., being deformed) in the thickness direction (e.g., beyond a predetermined thickness and/or volume). The abutment plates 52 prevent a volume change due to the expansion of the first accommodation chamber 26 and the second accommodation chamber 28 in the thickness direction, thereby suppressing the variations in the amount of the introduced platelet product.

The frame body 54 of the first frame member 50a and the frame body 54 of the second frame member 50b sandwich and hold a peripheral edge portion of the collection bag 14 in the thickness direction. The frame bodies 54 keep a planar shape of the collection bag 14, thereby improving the handleability of the collection kit 10A. In addition, the frame member 50 having the frame bodies 54 prevents a volume change due to the deformation of the first accommodation chamber 26 and the second accommodation chamber 28, and suppresses the variations in the amount of the introduced platelet product.

Note that a clamp structure that closes the tubular portion 31 may be integrally attached to the frame member 50 in the collection kit 10A of the present embodiment. In this case, work of attaching the clamp 42, prepared separately, to the tubular portion 31 becomes unnecessary, and the operability and handleability are further improved.

The above disclosure is summarized as follows.

According to at least one aspect of the present disclosure, a collection kit (10) is provided including: an inlet tube (12) to which a medical bag (94) accommodating an object to be collected is connected; a collection bag (14) which is connected to a downstream side of the inlet tube, includes a plurality of accommodation chambers (26, 28) formed by overlapping and welding two flexible sheets (22), and accommodates a predetermined amount of the object to be collected in each of the accommodation chambers; a plurality of outlet ports (34, 36) which are connected to the accommodation chambers, respectively, and allow the object to be collected to flow out from the accommodation chambers; and adapters (21a, 21b) which are connected to downstream sides of the outlet ports, respectively, and connect culture bottles (90, 92) and the outlet ports by the culture bottles being attached to the adapters, respectively, wherein the collection bag includes a connection flow path (30) connecting the accommodation chambers adjacent to each other, and the connection flow path is integrally formed with the accommodation chambers by the two flexible sheets, and has a tubular portion (31) formed in a tubular shape by cutting out the flexible sheets around the tubular portion between the accommodation chambers adjacent to each other.

The collection kit has a structure in which the collection bag is integrally formed using the two flexible sheets, and thus can be manufactured at low cost. In addition, the collection kit can reliably separate the adjacent accommodation chambers by closing the tubular portion of the collection bag with a clamp or the like, and can suppress the variations in the amount of the introduced platelet product.

In the collection kit described above, the plurality of accommodation chambers may include a first accommodation chamber (26) having an upper portion to which the inlet tube is connected, and a second accommodation chamber (28) connected to the first accommodation chamber via the connection flow path, and an exhaust valve (20) that exhausts air inside the first accommodation chamber and the second accommodation chamber may be connected to an upper portion of the second accommodation chamber. This collection kit can smoothly introduce the platelet product by discharging the air inside the first accommodation chamber and the second accommodation chamber through the exhaust valve.

In the collection kit described above, an upstream end (30a) of the connection flow path may be connected to the upper portion of the first accommodation chamber, and a downstream end (30g) of the connection flow path may be connected to a lower portion of the second accommodation chamber. In this collection kit, the platelet product can be smoothly introduced without leaving air in the first accommodation chamber and the second accommodation chamber.

In the collection kit described above, the plurality of outlet ports may include a first outlet port (34) connected to a lower portion of the first accommodation chamber and a second outlet port (36) connected to a lower portion of the second accommodation chamber. In this collection kit, the platelet product in the first accommodation chamber and the platelet product in the second accommodation chamber can be introduced into the culture bottles from the different outlet ports, respectively, and thus, work of transferring the platelet product between different accommodation chambers becomes unnecessary, and the operability is excellent.

The collection kit described above may further include a frame member (50) that holds peripheral edge portions of the flexible sheets and holds the flexible sheets in a planar shape. Since this collection kit can keep the collection bag in a planar shape, work of maintaining the collection bag in a planar shape during use becomes unnecessary, and thus, the handleability is excellent.

In the collection kit described above, the frame member may include: a frame body (54) that holds the peripheral edge portions of the flexible sheets; and an abutment plate (52) that abuts on the accommodation chambers in a thickness direction of the flexible sheets to prevent the accommodation chambers from expanding in the thickness direction. This collection kit can suppress the variations in the amount of the introduced platelet product by suppressing the expansion of the accommodation chambers in the thickness direction.

According to another aspect of the present disclosure, a collection method is provided using the collection kit described above, the collection method including: a step of connecting the medical bag to the inlet tube; a step of introducing the object to be collected into the accommodation chambers of the collection bag through the inlet tube; a step of separating the medical bag from the inlet tube; a step of closing the tubular portion to prevent the object to be collected from moving between the plurality of accommodation chambers; and a step of connecting the culture bottles to the adapters and injecting the object to be collected into the culture bottles. According to this collection method, work efficiency of a culture test of the platelet product can be improved.

In the collection method described above, the tubular portion may be closed by a clamp, ultrasonic sealing, or high frequency sealing in the step of closing the tubular portion to prevent the object to be collected from moving between the plurality of accommodation chambers.

Note that the present invention is not limited to the disclosure, and various configurations can be adopted without departing from the gist of the present invention.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A collection kit comprising:
an inlet tube to which a medical bag accommodating an object to be collected is connected;
a collection bag which is connected to a downstream side of the inlet tube, includes a plurality of accommodation chambers formed by overlapping and welding two flexible sheets, and accommodates a predetermined amount of the object to be collected in each of the accommodation chambers;
a plurality of outlet ports which are connected to the accommodation chambers, respectively, and allow the object to be collected to flow out from the accommodation chambers; and
adapters which are connected to downstream sides of the outlet ports, respectively, and connect culture bottles and the outlet ports by the culture bottles being attached to the adapters, respectively, wherein
the collection bag includes a connection flow path connecting the accommodation chambers adjacent to each other, and
the connection flow path is integrally formed with the accommodation chambers by the two flexible sheets, and has a tubular portion formed in a tubular shape by cutting out the flexible sheets around the tubular portion between the accommodation chambers adjacent to each other.

2. The collection kit according to claim 1, wherein the plurality of accommodation chambers include a first accommodation chamber having an upper portion to which the inlet tube is connected, and a second accommodation chamber connected to the first accommodation chamber via the connection flow path,
the collection kit further comprising an exhaust valve which is connected to an upper portion of the second accommodation chamber and exhausts air inside the first accommodation chamber and the second accommodation chamber.

3. The collection kit according to claim 2, wherein an upstream end of the connection flow path is connected to the upper portion of the first accommodation chamber, and a downstream end of the connection flow path is connected to a lower portion of the second accommodation chamber.

4. The collection kit according to claim 2, wherein the plurality of outlet ports include a first outlet port connected to a lower portion of the first accommodation chamber and a second outlet port connected to a lower portion of the second accommodation chamber.

5. The collection kit according to claim 1, further comprising a frame member which holds peripheral edge portions of the flexible sheets and holds the flexible sheets in a planar shape.

6. The collection kit according to claim 5, wherein the frame member includes: a frame body that holds the peripheral edge portions of the flexible sheets; and an abutment plate that abuts on the accommodation chambers in a thickness direction of the flexible sheets to prevent the accommodation chambers from expanding in the thickness direction.

7. A collection method using the collection kit according to claim 1, the collection method comprising:
a step of connecting the medical bag to the inlet tube;
a step of introducing the object to be collected into the accommodation chambers of the collection bag through the inlet tube;
a step of separating the medical bag from the inlet tube;
a step of closing the tubular portion to prevent the object to be collected from moving between the plurality of accommodation chambers; and
a step of connecting the culture bottles to the adapters and injecting the object to be collected into the culture bottles.

8. The collection method according to claim 7, wherein
the tubular portion is closed by a clamp, ultrasonic sealing, or high frequency sealing in the step of closing the tubular portion to prevent the object to be collected from moving between the plurality of accommodation chambers.

9. A collection kit, comprising:
a collection bag extending a width distance from a first side of the collection bag to a second side of the collection bag and extending a height distance from an upper side of the collection bag to a lower side of the collection bag, the collection bag comprising:
a first flexible sheet comprising a first shape extending along the width distance and the height distance;
a second flexible sheet comprising a second shape extending along the width distance and the height distance, the second flexible sheet arranged overlapping and in contact with the first flexible sheet in a thickness direction,
a first accommodation chamber arranged between the first flexible sheet and the second flexible sheet, the first accommodation chamber disposed adjacent the first side of the collection bag;
a second accommodation chamber arranged between the first flexible sheet and the second flexible sheet, the second accommodation chamber disposed adjacent the second side of the collection bag; and
a connection flow path comprising an upstream end and a downstream end, the upstream end fluidly interconnected to the first accommodation chamber at an exit point of the first accommodation arranged closer to the upper side of the collection bag than the lower side of the collection bag, the downstream end fluidly interconnected to the second accommodation chamber at an inflow point of the second accommodation arranged closer to the lower side of the collection bag than the upper side of the collection bag, wherein the connection flow path fluidly interconnects the first accommodation chamber with the second accommodation chamber; and
an inlet tube that is operatively connected to the first accommodation chamber, the inlet tube configured to attach to a medical bag comprising an object to be collected.

10. The collection kit according to claim 9, further comprising:
an inlet port fluidly interconnected with the first accommodation chamber and arranged adjacent the upper side of the collection bag, wherein inlet port is fluidly interconnected with the inlet tube;
a first outlet port fluidly interconnected with the first accommodation chamber and arranged adjacent the lower side of the collection bag; and
a second outlet port fluidly interconnected with the second accommodation chamber and arranged adjacent the lower side of the collection bag.

11. The collection kit according to claim 10, further comprising:
an exhaust port fluidly interconnected to the second accommodation chamber and arranged adjacent the upper side of the collection bag.

12. The collection kit according to claim 11, wherein the connection flow path comprises a connection region arranged between the first side of the collection bag and the second side of the collection bag, and wherein the collection bag further comprises:
a first recess arranged between the upper side of the collection bag and the connection region of the connection flow path; and
a second recess arranged between the lower side of the collection bag and the connection region of the connection flow path.

13. The collection kit according to claim 12, further comprising:
a first adapter interconnected to the first outlet port, the first adapter comprising a first needle configured to selectively fluidly interconnect with a first culture bottle attached thereto; and
a second adapter interconnected to the second outlet port, the second adapter comprising a second needle configured to selectively fluidly interconnect with a second culture bottle attached thereto.

14. The collection kit according to claim 13, further comprising:
a clamp attached to the connection region of the connection flow path, wherein the clamp is movable between an open state and a closed state, wherein, in the open state, the first accommodation chamber and the second accommodation chamber are maintained in fluid communication, and wherein, in the closed state, the first accommodation chamber and the second accommodation chamber are not in fluid communication.

15. The collection kit according to claim 10, further comprising:
an exhaust valve operatively connected to the exhaust port, the exhaust valve configured to allow passage of gas from the collection bag and prevent passage of liquid from the collection bag.

16. The collection kit according to claim 10, further comprising:
a frame that holds peripheral edge portions of the collection bag and holds the collection bag in a planar shape.

17. The collection kit according to claim 16, wherein the frame comprises:
a first frame body portion; and
a second frame body portion attached to the first frame body portion, wherein the first frame body portion and the second frame body portion comprise recesses that accommodate the first accommodation chamber and the second accommodation chamber of the collection bag in the thickness direction.

18. The collection kit according to claim 17, wherein the peripheral edge portions of the collection bag are held and disposed between abutment surfaces of the first frame body portion and the second frame body portion.

19. The collection kit according to claim 17, wherein the recesses define a space inside the frame that prevent a change in volume of the first accommodation chamber and the second accommodation chamber beyond a predetermined volume.

20. The collection kit according to claim 17, wherein the frame comprises a cutout arranged around a portion of the connection flow path between the first side of the collection bag and the second side of the collection bag.
